(19) United States
(12) Patent Application Publication
(10) Pub. No.: US 2016/0067267 A1
(43) Pub. Date: Mar. 10, 2016

(54) MYO-INOSITOL TRISPYROPHOSPHATE AS AN ANTI-OBESITY AGENT

(71) Applicant: NormOxys, Inc., Brighton, MA (US)

(72) Inventors: Ruth Greferath, Kehl (DE); Jean-Marie Lehn, Strasbourg (FR); Yves Claude Nicolau, Newton, MA (US)

(73) Assignee: NormOxys, Inc., Brighton, MA (US)

(21) Appl. No.: 14/775,763

(22) PCT Filed: Mar. 14, 2014
(86) PCT No.: PCT/US14/28130

§ 371 Date: Sep. 14, 2015

Related U.S. Application Data
(60) Provisional application No. 61/792,367, filed on Mar. 15, 2013.

(51) Int. Cl. A61K 31/6615; A61K 9/00

(52) U.S. Cl. CPC A61K 31/6615; A61K 9/0053; A61K 9/0019

(57) ABSTRACT

The present invention provides for novel treatments of obesity and overweight and related disorders. The invention provides, in part, method of treatment comprising, or uses of inositol-tripyrophosphate (ITPP) in these disease states.

### PRIORITY

This application claims the benefit of priority to U.S. Provisional Application No. 61/792,367, filed Mar. 15, 2013, the contents of which are hereby incorporated by reference in their entirety.

### FIELD OF THE INVENTION

The present invention relates to the treatment and prevention of weight-related diseases (including obesity and overweight).

### BACKGROUND

Obesity and overweight and related diseases, are major health problems in the modern world.

Obesity refers to the medical condition in which adipose tissue (body fat) is systemically accumulated as a result of, for example, energy intake that is higher than energy expenditure over a long period of time. Obesity may result in various associated diseases, such as arteriosclerosis.

A number of therapeutic methods for preventing obesity have been attempted, but these methods have proven unsatisfactory. For instance, some drug therapies using hormones or excitometabolics suffer from non-selective decomposition of proteins with fats. In addition, anorectics and digestive enzyme-inhibitors suffer from a problem of side-effects such as nervous symptoms and diarrhea.

Therefore, there remains a need for more effective and accessible methods of treating obesity, and related diseases.

### SUMMARY OF THE INVENTION

Accordingly, the present invention provides new methods and uses for the treatment and/or prevention of obesity, and related diseases, comprising inositol-tripyrophosphate (ITPP, and compounds related to ITPP).

In some aspects, the present invention provides for methods of treatment comprising administering ITPP (and compounds related to ITPP) and/or uses of ITPP (and compounds related to ITPP) in the treatment of or manufacture of a medicament for obesity and overweight, and related conditions. For example, in some embodiments, the present invention provides for the use of ITPP (and compounds related to ITPP) to induce weight loss and/or to prevent weight gain by, for example, affecting adipose tissue. In some aspects, the present invention provides for methods of treatment comprising administering ITPP (and compounds related to ITPP) and/or uses of ITPP (and compounds related to ITPP) for inducing weight loss or preventing weight gain, in a patient that has undertaken or will undertake a surgery of the digestive system; is greater than about 80-100 pounds overweight; has a BMI of greater than about 35; or has a health problem related to obesity. For example, in some embodiments, ITPP (and compounds related to ITPP) is a primary treatment of obesity and overweight, while in other embodiments, it is an adjuvant to other standard treatments, including, for example, restrictive surgeries and liposuctions. In some aspects, ITPP (and compounds related to ITPP) even induces weight loss or prevents weight gain in patients that do not substantially change caloric intake. For instance, a patient may consume upwards of about 2400 calories/day and still lose weight or not gain weight.

### BRIEF DESCRIPTION OF THE DRAWINGS

FIG. 1 shows the effects of ITPP on preventing weight gain. Thirty mice (10 mice each group) were used. HFD (high fat diet, diamonds) and ITPP solution were well tolerated (squares are HFD+ITPP). 70 days after the beginning of the experiment, the HFD-eating animals had gained weight approximately 10 g per animal (to a weight of ˜40 g per mouse), which is about one third more compared to the ITPP treatment group and control animals of this age (a weight of ˜30 g per mouse). Controls (normal food) are represented with triangles. Y axis is weight of Balb/c mice (g), X axis is time in days.

FIG. 2 also shows the effects of ITPP on preventing weight gain. Thirty mice (10 mice each group at day 30) were used. HFD and ITPP were tolerated very well. 70 days after the beginning of the experiment the HFD-eating animals drinking water had gained weight on the order of approximately 30% to a weight of ˜40 g per mouse (diamonds, top curve). While control mice (squares, bottom curve at day 30) and HFD/ITPP consumers (triangles, middle curve at day 30) did not gain significant weight and maintained a weight below around 30 g, the HFD/water Y axis is weight of Balb/c mice (g), X axis is time in days.

FIG. 3 shows the effects of overweight Balb/c-mice (5 each group, taken from the second experiment) drinking either pure water (squares) or water with ITPP 10 g per liter (diamonds). Both groups received normal mouse food. ITPP-treated mice lost considerably more weight than the untreated group. Y axis is weight of Balb/c mice (g), X axis is time in weeks.

### DETAILED DESCRIPTION

The present invention is based, in part on the discovery that ITPP is useful in the treatment of obesity or overweight. For example, in some aspects, the present invention shows that ITPP prevents weight gain without caloric reduction and causes weight loss or substantially reduces the trajectory of weight gain.

In some aspects, the present invention provides for methods of treatment comprising administering ITPP and/or uses of ITPP in the treatment of or manufacture of a medicament for obesity and overweight, and related conditions.

ITPP (“inositol-tripyrophosphate” or “inositol hexaphosphate trispyrophosphate” or “IHP-tripyrophosphate” or “OXY111A”) refers to an inositol hexaphosphate with three internal pyrophosphate rings, as described in, for example, U.S. Pat. No. 8,178,514, the contents of which are hereby incorporated by reference in their entirety. In various embodiments, acids and salts of ITPP (and compounds related to ITPP) are used. In some embodiments, ITPP (and compounds related to ITPP) is an anion. The counterpart species to ITPP may be a counterion and the combination of ITPP with a counterion is an acid or salt. Counter ions of ITPP (and compounds related to ITPP) may include, but are not limited to, cationic hydrogen species including protons; monovalent inorganic cations including lithium, sodium, and potassium; divalent inorganic cations including magnesium, calcium, manganese, zinc, copper and iron; polyvalent inorganic cations including iron; quaternary nitrogen species including ammonium, cycloheptyl ammonium, cyclooctyl ammonium, N,N-dimethylcyclohexyl ammonium, and other organic ammonium cations; sulfonium species including triethylsulfonium and other organic sulfonium compounds; organic cations including pyridinium, piperidinium, piperazinium, quinuclidinium, pyrrolium, tripiperazinium, and other organic cations; polymeric cations including oligomers, polymers, peptides, proteins, positively charged ionomers, and other macromolecular species that possess sulfonium, quaternary nitrogen and/or charged organometallic species in pendant groups, chain ends, and/or the backbone of the polymer. An illustrative ITPP salt is the monocalcium tetrasodium salt of ITPP or a mixture of sodium ITPP and calcium ITPP that contains 15-25 mol % calcium and 75-85 mol % sodium.

The invention is not limited to pairings that are purely ionic; indeed, it is well-known in the art that paired ions may evidence some degree of covalent or coordinate bond characteristic between the two components of the pair. The ITPP (and compounds related to ITPP) acids and salts of the invention compositions may comprise a single type of counterion or may contain mixed counterions, and may optionally contain a mixture of anions of which ITPP is one. The compositions may optionally include crown ethers, cryptands, and other species capable of chelating or otherwise complexing the counterions. The compositions may likewise optionally include acidic macrocycles or other species that are capable of complexing the ITPP (and compounds related to ITPP) through hydrogen bonds or other molecular attractions.

ITPP (and compounds related to ITPP), in various embodiments, may be present in various isomers. In some embodiments, ITPP is myo-inositol (cis-1,2,3,5-trans-4,6-cyclohexanehexyl), while the invention also provides for any inositol isomer in the ITPP (e.g. tripyrophosphates of the naturally occurring scyllo-, chiro-, muco-, and neo-inositol isomers, as well as those of the allo, epi-, and cis-inositol isomers).

Methods of making acids and salts of ITPP are described in U.S. Pat. No. 7,084,115, the entire contents of which is incorporated herein by reference. Also, ITPP may be formed in vive from a prodrug, such as by enzymatic cleavage of an ester (such as an alkyl ester) or by displacement of a leaving group such as a tolylsulfonyl group.

Also provided are methods making a pharmaceutical composition of ITPP (and compounds related to ITPP) by mixing a sodium and calcium salt of ITPP (and compounds related to ITPP) and a pharmaceutically acceptable adjuvant, diluent, carrier, or excipient thereof. In some embodiments, the mixture of the sodium and calcium salt of ITPP is obtained by mixing myoinositol tripyrophosphate-sodium salt with CaCl₂.

In some embodiments, the present invention also encompasses methods of treatment and uses as described herein for compounds related to ITPP, as alluded to above. For example, in some embodiments, the invention provides for methods of treatment and uses comprising a pharmaceutical composition comprising a compound represented by structure: nC⁺Aⁿ⁻, wherein: C⁺ represents independently for each occurrence an alkali metal cation (e.g., a sodium ion, a lithium ion, a potassium ion, etc.), an alkaline earth cation (e.g. a magnesium ion or calcium ion), or an ammonium cation; A represents an anionic moiety (e.g. phosphorylated inositol; IHP, wherein two phosphate groups of the IHP form an internal pyrophosphate ring; IHP, wherein 4 phosphate groups of said IHP form two internal pyrophosphate rings; IHP, wherein 6 phosphate groups of said IHP form three internal pyrophosphate rings); and n is an integer in the range of 1 to 10 inclusive (e.g. 1, or 2, or 3, or 4, or 5, or 6, or 7, or 8, or 9, or 10). In various embodiments, C⁺ is a sodium ion and Aⁿ⁻ is a phosphorylated inositol; or C⁺ is a sodium ion and Aⁿ⁻ is a phosphorylated inositol, wherein the phosphorylated inositol has one internal pyrophosphate ring; or C⁺ is a sodium ion and Aⁿ⁻ is a phosphorylated inositol, wherein the phosphorylated inositol has two internal pyrophosphate rings; or C⁺ is a sodium ion and Aⁿ⁻ is a phosphorylated inositol, wherein the phosphorylated inositol has three internal pyrophosphate rings; or C⁺ is a sodium ion and Aⁿ⁻ is IHP; or C⁺ is a sodium ion and Aⁿ⁻ is IHP, wherein two phosphate groups of said IHP form an internal pyrophosphate ring; or C⁺ is a sodium ion and Aⁿ⁻ is IHP, wherein 4 phosphate groups of said IHP form two internal pyrophosphate rings; or C⁺ is a sodium ion and Aⁿ⁻ is IHP, wherein the 6 phosphate groups of said IHP form three internal pyrophosphate rings.

In still other embodiments, compounds related to ITPP are those recited in US Patent Publication No. 2008/0200437, International Patent Publication No. WO 2012/045009, and U.S. patent application Ser. No. 13/897,113, the contents of which are hereby incorporated by reference in their entireties. For instance, in some embodiments, compounds related to ITPP are based on ITPP, which is altered to have one or more of: a derivatized phosphate group hydroxyl (e.g. selected from alkoxy (—OR) or acyloxy (—OCOR), where R is selected from alkyl, aryl, acyl, aralkyl, alkenyl, alkynyl, heterocyclyl, carbocycle, amino, acylamino, amido, alkylthio, sulfonate, alkoxyl, sulfonyl, or sulfoxide, or a salt derivative); the inositol in various conformations (such as, for example, cis-inositol, epi-inositol, allo-inositol, muco-inositol, neo-inositol, scyllo-inositol, (+) chiro-inositol, or (−) chiro-inositol); a substitution of inositol for another moiety (e.g. a compound that is a polyphosphate or pyrophosphate derivative of a mono-, di- or oligosaccharide containing a pyranose or furanose unit (e.g. glucose, mannose, or galactose, sucrose or lactose); or a pharmaceutical acceptable salt, stereoisomer, anomer, solvate, and hydrate thereof.

In some embodiments, the compound related to ITPP is 1,6:3,4-Bis-[O-(2,3-dimethoxybutane-2,3-diyl)]-2,5-di-O-methyl-myo-inositol; 2,5-Di-O-methyl-myo-inositol; Octabenzyl 1,3,4,6-(2,5-di-O-methyl-myo-inosityl)tetrakisphosphate; Tetrasodium 1,3,4,6-(2,5-di-O-methyl-myo-inosityl)tetrakisphosphate; 1,6:3,4-Bis-[O-(2,3-dimethoxybutane-2,3-diyl)]-2,5-di-O-ethyl-myo-inositol; 2,5-Di-O-ethyl-myo-inositol; Octabenzyl 1,3,4,6-(2,5-di-O-ethyl-myo-inosityl)tetrakisphosphate; Tetrasodium 1,3,4,6-(2,5-di-O-ethyl-myo-inosityl)tetrakisphosphate; 1,6:3,4-Bis-[O-(2,3-dimethoxybutane-2,3-diyl)]-2,5-di-O-butyl-myo-inositol; 2,5-Di-O-butyl-myo-inositol; Octabenzyl 1,3,4,6-(2,5-di-O-butyl-myo-inosityl)tetrakisphosphate; Tetrasodium 1,3,4,6-(2,5-di-O-butyl-myo-inosityl)tetrakisphosphate; 2,5-Di-O-benzyl-1,6:3,4-bis-[O-(2,3-dimethoxybutane-2,3-diyl)]-myo-inositol; 2,5-Di-O-benzyl-myo-inositol; Octabenzyl 1,3,4,6-(2,5-di-O-benzyl-myo-inosityl)tetrakisphosphate; Tetrasodium 1,3,4,6-myo-inosityl tetrakisphosphate; Hexabenzyl 1,3,5-(2,4,6-tri-O-butyryl-myo-inosityl)trisphosphate; Hexasodium 1,3,5-(2,4,6-tri-O-butyryl-myo-inosityl)trisphosphate; Orthoformate of myo-inositol 2,4,6-tris(dibenzyl phosphate); Orthoformate of hexasodium myo-inositol 2,4,6-trisphosphate; scyllo-inositol hexakis(dibenzyl phosphate); Hexatriethylammonium scyllo-inositol hexakisphosphate; Hexatriethylammonium scyllo-inositol 1,2:3,4:5,6-trispyrophosphate; or Hexasodium scyllo-inositol 1,2:3,4:5,6-trispyrophosphate.

In some embodiments, the compound related to ITPP is 1-O-methyl-α-glucose 2,3,4-trisphosphate, 1-O-methyl-α-mannose 2,3,4-trisphosphate, α-glucose 1,2,3,4-tetrakisphosphate, β-glucose 1,2,3,4-tetrakisphosphate, α-mannose 1,2,3,4-tetrakisphosphate, β-mannose 1,2,3,4-tetrakisphosphate, α-galactose 1,2,3,4-tetrakisphosphate, β-galactose 1,2,3,4-tetrakisphosphate, 1-O-methyl-α-glucose tetrakisphosphate, 1-O-methyl-α-mannose tetrakisphosphate, α-glucose pentakisphosphate, α-mannose pentakisphosphate, α-galactose pentakisphosphate, lactose octakisphosphate, sucrose octakisphosphate, or 1-O-methyl-α-glucose bispyrophosphate).

In some embodiments, the compound related to ITPP is selected from diethyl-2,3-bisphospho-L-tartrate tetrasodium and di sodium salt;dibutyl-2,3-bisphospho-L-tartrate tetrasodium salt and dibutyl-cyclo-2,3-bisphospho-L-tartrate disodium salt; 2,3-bisphospho-L-tartrate hexasodium salt; tetrasodium dimethyl-meso-galactarate-2,3,4,5-tetrakisphosphate and its bispyrophosphates; tetrasodium meso-erythritol-1,2,3,4-tetrakisphosphate and its bispyrophosphates; tetrasodium pentaerythritol-2,3,4,5-tetrakisphosphate and its bispyrophosphate; tetrasodium 2,5-anhydro-D-mannitol-1,3,4,6-tetrakisphosphate and its bispyrophosphates; Diethyl-2,3-bis(dibenzylphospho)-L-tartrate; Diethyl-2,3-bisphospho-L-tartrate tetrasodium salt; Diethyl-2,3-bisphospho-L-tartrate disodium salt;Dibutyl-2,3-bis(dibenzylphospho)-L-tartrate; Dibutyl-2,3-bisphospho-L-tartrate tetrasodium salt; Dibutyl-2,3-bisphospho-L-tartrate ditriethylammonium salt; Dibutyl-cyclo-2,3-bisphospho-L-tartrate ditriethylammonium salt; Dibutyl-cyclo-2,3-bisphospho-L-tartrate disodium salt; Dibenzyl-2,3-bis(dibenzylphospho)-L-tartrate; 2,3-Bisphospho-L-tartrate hexasodium salt; Dimethyl-2,3,4,5-tetrakis(dibenzylphospho)-meso-galactarate; Tetrasodium dimethyl-meso-galactarate-2,3,4,5-tetrakisphosphate; Tetrasodium dimethyl-meso-galactarate bispyrophosphates; 1,2,3,4-Tetrakis(dibenzylphospho)-meso-erythritol; 1,2,3,4-Tetrakisphospho-meso-erythritol tetrasodium salt; Tetrasodium meso-crythritol bispyrophosphate; 1,3,4,5-Tetrakis(dibenzylphospho)pentaerythritol; Tetrasodium pentaerythritol 1,3,4,5-tetrakisphosphate; Tetrasodium pentaerythritol (1,3):(4,5)-bispyrophosphate; 1,3,4,6-Tetrakis(dibenzylphospho) 2,5-anhydro-D-mannitol; Tetrasodium 2,5-anhydro-D-mannitol 1,3,4,6-tetrakisphosphate; and Tetrasodium 2,5-anhydro-D-mannitol bispyrophosphate.

In some aspects, the present invention provides for methods of treatment comprising administering ITPP (and compounds related to ITPP) and/or uses of ITPP (and compounds related to ITPP) in the treatment of, or manufacture of a medicament for, obesity and overweight, and related conditions. In some aspects, the present invention provides a method for treating or preventing obesity, comprising administering an effective amount of ITPP (and compounds related to ITPP) to a patient in need thereof. In some aspects, the present invention provides a method for weight management, comprising administering an effective amount of ITPP (and compounds related to ITPP) to induce weight loss and/or to prevent weight gain in a patient in need thereof.

In some aspects, the present invention relates to a method for inducing weight loss or preventing weight gain (or treating or preventing obesity or inducing weight loss or preventing weight gain in a patient that does not substantially change caloric intake), comprising administering an effective amount of ITPP (and compounds related to ITPP) to a patient that: has undertaken or will undertake a surgery of the digestive system; is greater than about 80-100 pounds overweight; has a BMI of greater than about 35; or has a health problem related to obesity.

In various embodiments, the surgery of the digestive system is one or more of those classified under ICD-9-CM [42-54]: Operations on the Digestive System and therefore may include [42] Operations on esophagus; [43] Incision and excision of stomach; [44] Other operations on stomach; [45] Incision, excision, and anastomosis of intestine; [46] Other operations on intestine; [47] Operations on appendix; [48] Operations on rectum, rectosigmoid and perirectal tissue; [49] Operations on anus; [50] Operations on liver; [51] Operations on gallbladder and biliary tract; [52] Operations on pancreas; [53] Repair of hernia; and [54] Other operations on abdominal region.

In some embodiments, the surgery of the digestive system is one or more of a restrictive surgery and/or malabsorptive procedure, including, for example, vertical banded gastroplasty (VBG, e.g. stomach stapling); gastric banding (e.g. LAP-BAND or REALIZE); sleeve gastrectomy; gastric bypass surgery (e.g. Roux-en-Y gastric bypass), biliopancreatic diversion and a cosmetic surgery (e.g. liposuction, such as, for example, suction-assisted liposuction (SAL); ultrasound-assisted liposuction (UAL); power-assisted liposuction (PAL); twin-cannula (assisted) liposuction (TCAL or TCL); external ultrasound-assisted liposuction (XUAL or EUAL); water-assisted liposuction (WAL); laser assisted liposuction; tumescent liposuction; and cryolipolysis).

In some embodiments, the health problem related to obesity is selected from cardiovascular diseases (e.g. high cholesterol, hypercholesterolemia, low HDL, high HDL, hypertension, coronary artery disease, heart failure), sleep apnea (including obstructive sleep apnea), osteoarthritis, thyroid problems, dementia, gout, asthma, gastroesophageal reflux disease, and chronic renal failure. In some embodiments, the health problem related to obesity is heart disease, sleep apnea, or high cholesterol.

In some aspects, the present invention provides for uses and methods for inducing weight loss or preventing weight gain, comprising administering an effective amount of ITPP (and compounds related to ITPP) to a patient in need thereof; wherein the patient does not substantially change caloric intake. In some embodiments, the caloric intake is high, relative to guidelines, such as the USDA tables. In some embodiments, the patient's caloric intake is 2000-10000 calories/day, or greater than about 2000 calories/day, or about 2200 calories/day, or about 2400 calories/day, or about 2600 calories/day, or about 2800 calories/day, or about 3000 calories/day, or about 3200 calories/day, or about 3400 calories/day, or about 3600 calories/day, or about 3800 calories/day, or about 4000 calories/day, or about 5000 calories/day, or about 6000 calories/day. In various embodiments, the patient has a high caloric intake and does not gain weight or even loses weight. Therefore, the present invention provides for an effect without life style changes that often reduce patient adherence (e.g. failed dieting). In some embodiments, the patient's caloric intake is not restricted by more than about 20%, or not by more than about 10%, or not by more than about 5% of the patient's caloric intake at the start of treatment. In some embodiments, a high proportion of the patient's caloric intake is “empty calories,” i.e. calories from solid fats and/or added sugars. In some embodiments, greater than about 15%, or 20%, or 25%, or 30%, or 35%, or 50% of the patient's caloric intake is empty calories. Even in these embodiments, a patient may not gain weight or even lose weight.

In various embodiments, the patient of the present invention is overweight or obese. In some embodiments, the patient of the present invention suffers from central obesity. In some embodiments, the obesity of one of simple obesity (alimentary obesity; usually resulting from consumption of more calories than the body can utilize), secondary obesity (usually resulting from an underlying medical condition, such as, for example, Cushing's syndrome and polycystic ovary syndrome), and childhood obesity. In some embodiments, the obesity is classified as: Class I, which includes a BMI between 30 and 34.99; Class II, which includes BMIs of 35 to 39.99; and Class 111, which includes a BMI of over 40. Further, the present invention provides for obesity of any of classes I, II, or III that is further classified as severe, morbid, and super obesity. In some embodiments, the patient is at risk of further weight gain, as assessed by, for example, daily caloric intake.

In various embodiments, the weight management/weight loss/anti-obesity effects of ITPP (and compounds related to ITPP) can be assessed using various techniques and indices. In various embodiments, assessment before, during, and after treatment is undertaken. In some embodiments, body mass index (BMI), a measure of a person's weight taking into account height, may be used. In various embodiments, a patient described herein has a BMI that provides an “overweight” classification, i.e. 25-29.9, such as, for example, about 25, or about 25.5, or about 26, or about 26.5, or about 27, or about 27.5, or about 28, or about 28.5, or about 29, or about 29.5. In various embodiments, a patient described herein has a BMI that provides an “obese” classification, i.e. greater than 30, such as, for example, about 30, or about 31, or about 32, or about 33, or about 34, or about 35, or about 36, or about 37, or about 38, or about 39, or about 40, or about 50. In some embodiments, body volume index (BVI) is used. BVI uses 3D software to create an 31) image of a person so BVI can differentiate between people with the same BMI rating, but who have a different shape and different weight distribution. BVI measures where a person's weight and the fat are located on the body, rather than total weight or total fat content and places emphasis on the weight carried around the abdomen, commonly known as central obesity. In some embodiments, whole-body air displacement plethysmography (ADP) is used to assess the weight management/weight loss/anti-obesity effects of ITPP (and compounds related to ITPP). In some embodiments, simple weighing is used in the present invention. In some embodiments, skinfold calipers or “pinch test,” bioelectrical impedance analysis, hydrostatic weighing, or dual-energy X-ray absorptiometry (DEXA) may be used.

In some embodiments, simple circumferential measurement of the body may be used. In some embodiments, a patient of the present invention has a waist circumference exceeding about 35 inches, or about 36 inches, or about 37 inches, or about 38 inches, or about 39 inches, or about 40 inches, or about 41 inches, or about 42 inches, or about 43 inches, or about 44 inches, or about 45 inches, or about 46 inches, or about 47 inches, or about 48 inches, or about 50 inches, or about 55 inches, or about 60 inches. In some embodiments, the patient is male human with a waist circumference exceeding 40 inches. In some embodiments, the patient is a female human with a waist circumference exceeding 35 inches.

The methods of the invention may be used to treat humans having a body fat percentage above the recommended body fat percentage, i.e., at least in the “overweight” range, or at least in the “obese” range. The body fat percentage will differ between women and men. Specifically, for women, the methods of the invention may be used to treat a female human having a body fat percentage of at least about 25%, above 25%, at least about 32%, or above 32%. For men, the methods of the invention may be used to treat a male human having a body fat percentage of at least about 14%, above 14%, at least about 18%, above 18%, at least about 25%, or above 25%. Body fat percentage may be estimated using any method accepted in the art, including, for example, near infrared interactance, dual energy X-ray absorptiometry, body density measurement, bioelectrical impedance analysis, and the like.

The methods of the invention may be used to treat a patient who is a man that is greater than 100 pounds overweight and/or has waist circumference exceeding 40 inches. The methods of the invention may be used to treat a patient who is a woman that is greater than 80 pounds overweight and/or waist circumference exceeding 35 inches.

In some embodiments, the invention provides for ITPP (and compounds related to ITPP) being used to treat and/or prevent certain disorders associated with being overweight. For example, ITPP (and compounds related to ITPP) find use in cardiovascular diseases (e.g. high cholesterol, hypercholesterolemia, low HDL, high HDL, hypertension, coronary artery disease, heart failure), sleep apnea (including obstructive sleep apnea), osteoarthritis, thyroid problems, dementia, gout, asthma, gastroesophageal reflux disease, and chronic renal failure.

In various embodiments, the ITPP (and compounds related to ITPP) administration and/or use prevents or reduces the growth of adipose tissue. In some embodiments. ITPP (and compounds related to ITPP) effects one or more of white adipose tissue (VAT) and brown adipose tissue (BAT), including, for example, visceral adipose tissue (VAT), abdominal subcutaneous adipose tissue (ASAT), or ectopic fat. Such an effect may be assessed by, for example, using any of the techniques described herein (e.g. BMI, weight for-stature indexes, skinfold measures, electrical bioimpedance analysis, etc.), as well as various imaging techniques, including computed tomography (CT), magnetic-resonance imaging (MRI, including transverse body scans), dual energy X-ray absorptiometry (DXA).

ITPP (and compounds related to ITPP) may also be used in combination with dietary therapy, behavioral therapy, physical therapy, exercise, and weight loss surgery, or a combination of two or more such therapies. In some embodiments, the subject is on a calorie restricted diet. In some embodiments, the subject engages in or is to engage in a physical exercise or physical therapy regimen. In some embodiments, the subject has undergone, or will undergo, weight loss surgery. In some embodiments, ITPP (and compounds related to ITPP) may be in combination with additional agents or may be administered to patient undergoing treatment with various agents.

For example, including, but not limited to, embodiments pertaining to obesity and/or weight reduction/loss, the additional agents may include one or more of orlistat (e.g. ALLI, XENICAL), loracaserin (e.g. BELVIQ), phentermine-topiramate (e.g. QSYMIA), sibutramine (e.g. REDUCTIL or MERIDIA), rimonabant (ACOMPLIA), exenatide (e.g. BYETTA), pramlintide (e.g. SYMLIN) phentermine, benzphetamine, diethylpropion, phendimetrazine, bupropion, and metformin.

Agents that interfere with the body's ability to absorb specific nutrients in food are among the additional agents, e.g. orlistat (e.g. ALLI, XENICAL), glucomannan, and guar gum. Agents that suppress apetite are also among the additional agents, e.g. catecholamines and their derivatives (such as phentermine and other amphetamine-based drugs), various antidepressants and mood stabilizers (e.g. bupropion and topiramate), anorectics (e.g. dexedrine, digoxin). Agents that increase the body's metabolism are also among the additional agents.

In some embodiments, additional agents may be selected from among appetite suppressants, neurotransmitter reuptake inhibitors, dopaminergic agonists, serotonergic agonists, modulators of GABAcrgic signaling, anticonvulsants, antidepressants, monoamine oxidase inhibitors, substance P (NKl) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, peroxisome proliferator-activated receptor (PPAR) modulators; dipeptidyl peptidase 4 (DPP-4) antagonists, agents for treating cardiovascular disease, agents for treating elevated triglyceride levels, agents for treating low HDL, agents for treating hypercholesterolemia, and agents for treating hypertension. Some agents for cardiovascular disease include statins (e.g. lovastatin, atorvastatin, fluvastatin, rosuvastatin, simvastatin and pravastatin) and omega-3 agents (e.g. LOVAZA, EPANOVA, VASCEPA, esterified omega-3's in general, fish oils, krill oils, algal oils). In some embodiments, additional agents may be selected from among amphetamines, benzodiazepines, sulfonyl ureas, meglitinides, thiazolidinediones, biguanides, beta-blockers. λCE inhibitors, diuretics, nitrates, calcium channel blockers, phenlermine, sibutramine, lorcaserin, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, repaglinide, nateglinide, glimepiride, metformin, pioglitazone, rosiglilazone, and sitagliptin.

In some embodiments, the ITPP (and compounds related to ITPP and/or additional agents) described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

In still other embodiments, the ITPP (and compounds related to ITPP) described herein may be modified to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

The ITPP (and compounds related to ITPP and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

Further, any ITPP (and compounds related to ITPP and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

The present invention includes the described ITPP (and compounds related to ITPP and/or additional agents) in various formulations. Any ITPP (and compounds related to ITPP and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the ITPP (and compounds related to ITPP and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the ITPP (and compounds related to ITPP and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any ITPP (and compounds related to ITPP and/or additional agents) described herein is formulated in accordance with routine procedures as a composition adapted for a mode of administration described herein.

In some embodiments, the administration of any ITPP (and compounds related to ITPP and/or additional agents) is any one of oral, intravenous, and parenteral. In other embodiments, routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and depends in-part upon the site of the medical condition. In most instances, administration results in the release of any agent described herein into the bloodstream.

Any ITPP (and compounds related to ITPP and/or additional agents) described herein can be administered orally. Such ITPP (and compounds related to ITPP and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment.

In one embodiment, any ITPP (and compounds related to ITPP and/or additional agents) described herein is formulated in accordance with routine procedures as a composition adapted for oral administration to humans. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any ITPP (and compounds related to ITPP and/or additional agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

The dosage of any ITPP (and compounds related to ITPP and/or additional agents) described herein as well as the dosing schedule can depend on various parameters, including, but not limited to, the disease being treated, the subject's general health, and the administering physician's discretion. Any agent described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional therapeutic agent, to a subject in need thereof. In various embodiments any agent described herein is administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart.

The amount of any ITPP (and compounds related to ITPP and/or additional agents) described herein that is admixed with the carrier materials to produce a single dosage can vary depending upon the subject being treated and the particular mode of administration. In vitro or in vivo assays can be employed to help identify optimal dosage ranges.

In general, the doses that are useful are known to those in the art. For example, doses may be determined with reference Physicians' Desk Reference, 66th Edition, PDR Network; 2012 Edition (Dec. 27, 2011), the contents of which are incorporated by reference in its entirety. In some embodiment, the present invention allows a patient to receive doses that exceed those determined with reference Physicians' Desk Reference.

The dosage of any ITPP (and compounds related to ITPP and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In a specific embodiment, dosage is influenced by a patient being overweight and/or obese and is adjusted to take into account concerns about dosing this population (e.g., increased volume of distribution for lipid soluble drugs, altered Phase II metabolism as glucuronidation and sulfonation can be enhanced and cause an increased clearance of drug, and increased renal clearance of drugs eliminated primarily through glomerular filtration).

In specific embodiments, the dosage of any ITPP (and compounds related to ITPP and/or additional agents) is about 0.2 to about 20 g/L when administered orally or about 4 g/mL when administered intravenously. More generally, when orally administered to a mammal, the dosage of any ITPP (and compounds related to ITPP and/or additional agents) described herein may be 0.001 mg/kg/day to 100 mg/kg/day, 0.01 mg/kg/day to 50 mg/kg/day, or 0.1 mg/kg/day to 10 mg/kg/day. When orally administered to a human, the dosage of any agent described herein is normally 0.001 mg to 1000 mg per day, 1 mg to 600 mg per day, or 5 mg to 30 mg per day. For administration of any ITPP (and compounds related to ITPP and/or additional agents) described herein by parenteral injection, the dosage is normally 0.1 mg to 250 mg per day, 1 mg to 20 mg per day, or 3 mg to 5 mg per day. Injections may be given up to four times daily. Generally, when orally or parenterally administered, the dosage of any agent described herein is normally 0.1 mg to 1500 mg per day, or 0.5 mg to 10 mg per day, or 0.5 mg to 5 mg per day. A dosage of up to 3000 mg per day can be administered.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any ITPP (and compounds related to ITPP and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any ITPP (and compounds related to ITPP and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject. Chronic, long-term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

The dosage regimen utilizing any ITPP (and compounds related to ITPP and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any ITPP (and compounds related to ITPP and/or additional agents) described herein can be administered in a single daily dose, or the total daily dosage can be administered in divided doses of two, three or four times daily. Furthermore, any ITPP (and compounds related to ITPP and/or additional agents) described herein can be administered continuously rather than intermittently throughout the dosage regimen.

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell.

In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

The invention provides kits that can simplify the administration of any agent described herein. An exemplary kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

This invention is further illustrated by the following non-limiting examples.

### EXAMPLES

### Example 1

### Anti-Obesity Effects of ITPP

Experiments were conducted in analogue ways. First, 30 Balb/c-mice, 10 mice in each group, were assessed. Group 1 was a control group; mice were fed with normal mouse food and water for the whole duration of the experiment (120 days). Group II was a group in which 10 mice received a high fat diet (HFD obtained from SAFE, Augy, France) and additionally normal food and water (former experiments showed that when normal mouse food additionally given to the high fat diet kept the animals more healthy over this rather long time. Therefore, we offered in each experiment with HFD also normal mouse food additionally). Group III was fed with high fat diet, normal food and, instead of normal water, animals drank an ITPP solution for the duration of the experiment of (ITPP). The concentration was 10 g ITPP per liter water, containing no calcium salt, pH˜6.8-7.2).

The HFD was 235HF (U8955 version 9) casein 20%, corn starch 13%, sucrose 24.3%, cellulose 5%, malto dextrin 2.2%, lard 25%, soja oil 2.5%, mineral 205B SAFE 7%, and vitamine 200 SAFE 1%.

| | 4 655 kcal/kg | | |
|---|---|---|---|
| | | energy level | % |
| Nutrients | Level % | kcal/kg | energy |
| Proteins | 17.0 | 680 | 14.6 |
| Lipids | 27.5 | 2475 | 53.2 |
| Carbohydrates | 37.5 | 1500 | 32.2 |

All food, water, and ITPP were given ad libitum. In 5 days intervals, body weights from each animal were recorded.

A second study was performed in the same way (again on 30 Balb/c-mice), the duration of the experiment was shorter (70 days), since a plateau was reached in gaining weight between day 50-70 of the experiment. As before, in 5 days intervals body weights of the Balb/c-mice were measured.

In a third study the group with the 10 mice which had gained weight (˜30%) was divided in two groups (5 animals per cage). For 1 month, one group drank water while the other one drank an ITPP-solution (10 g/L) for about 30 days until the date of observation. The food was normal mouse food as before (pellets from SAFE, France). The body weights of the mice were measured 4 times until the final day.

The high fat diet and the orally offered ITPP solution were accepted by the animals very well. No animal died or was suffering during the experiments over these two rather long periods (120 days and 70 days). Subject fur looked well and healthy, eyes were clear, and the vitality of all animals was normal.

Significant differences in the weights of the HFD eating mice which received water (control animals) and those who were fed with HFD and treated with ITPP (in water) additionally over time were observed. As early as day 10 of the experiment significant differences were observed. Animals of the ITPP drinking group did not increase their weight at all: they remained as those from the control group during the whole period of the experiment.

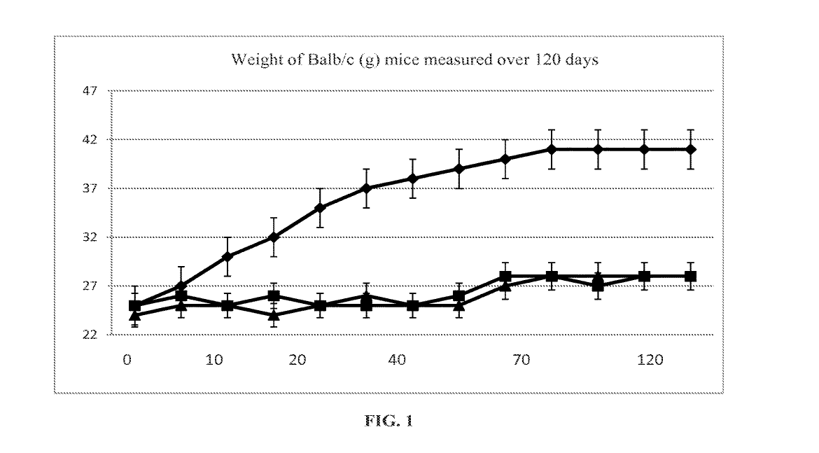

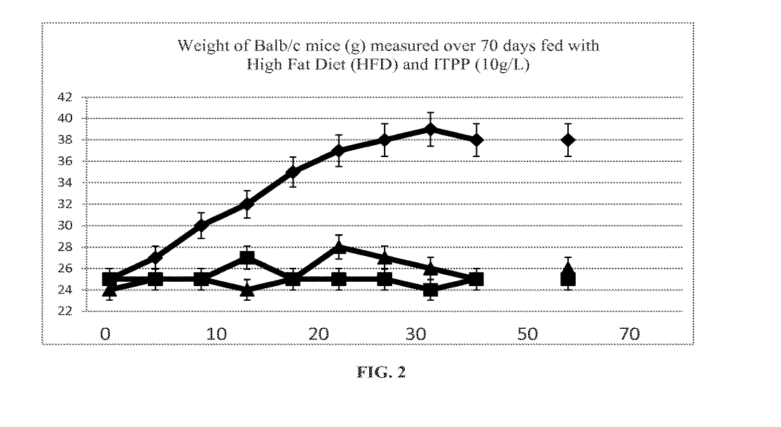

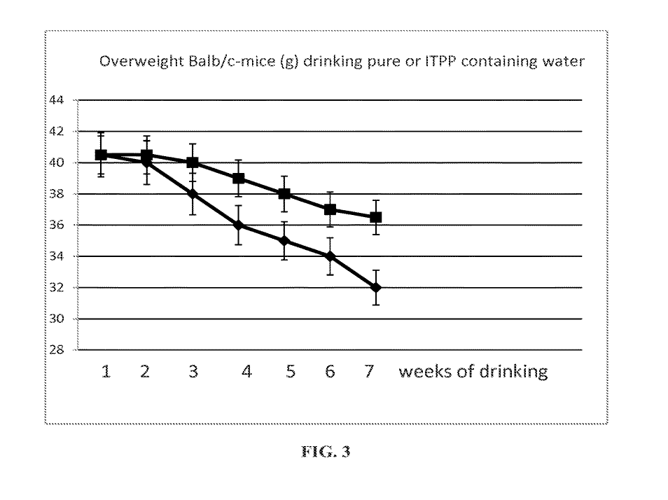

ITPP, orally applied to Balb/c mice not only helped individuals not gaining weight as shown in FIGS. 1 and 2. It also helped losing weight as FIG. 3 shows.

Specifically, FIG. 1 shows the effects of ITPP on preventing weight gain. Thirty mice (10 mice each group) were used. HFD (high fat diet, diamonds) and ITPP solution were well tolerated (squares are HFD+ITPP). 70 days after the beginning of the experiment, the HFD-eating animals had gained weight approximately 10 g per animal (to a weight of ˜40 g per mouse), which is about one third more compared to the ITPP treatment group and control animals of this age (a weight of ˜30 g per mouse). Controls (normal food) are represented with triangles. Y axis is weight of Balb/c mice (g), X axis is time in days.

FIG. 2 also shows the effects of ITPP on preventing weight gain. Thirty mice (10 mice each group at day 30) were used. HFD and ITPP were tolerated very well. 70 days after the beginning of the experiment the HFD-eating animals drinking water had gained weight on the order of approximately 30% to a weight of ˜40 g per mouse (diamonds, top curve). While control mice (squares, bottom curve at day 30) and HFD/ITPP consumers (triangles, middle curve at day 30) did not gain significant weight and maintained a weight below around 30 g, the HFD/water Y axis is weight of Balb/c mice (g), X axis is time in days.

FIG. 3 shows the effects of overweight Balb/c-mice (5 each group, taken from the second experiment) drinking either pure water (squares) or water with ITPP 10 g per liter (diamonds). Both groups received normal mouse food. ITPP-treated mice lost considerably more weight than the untreated group. Y axis is weight of Balb/c mice (g), X axis is time in weeks.

### DEFINITIONS

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents (e.g. ITPP (and compounds related to ITPP and/or additional agents) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a patient suffering from obesity or overweight provides a therapeutic benefit not only when the underlying condition is eradicated or ameliorated, but also when the patient reports a decrease in the severity or duration of the symptoms associated with the disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. In certain embodiments, the effect will result in a quantifiable change of two-fold, or three-fold, or four-fold, or five-fold, or ten-fold. Therapeutic benefit also includes halting or slowing the progression of the underlying disease or disorder or reduction in toxicity, regardless of whether improvement is realized.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method for treating or preventing obesity, comprising administering an effective amount of inositol-tripyrophosphate (ITPP) to a patient in need thereof.

2. A method for weight management, comprising administering an effective amount of ITPP to induce weight loss and/or to prevent weight gain in a patient in need thereof.

3. The method of claims 1 or 2, wherein the ITPP administration prevents or reduces the growth of adipose tissue.

4. The method of any of the above claims, wherein the patient is obese.

5. The method of claim 4, wherein the patient has a body mass index of greater than about 30.

6. The method of any of the above claims, wherein the patient is overweight.

7. The method of claim 6, wherein the patient has a body mass index of about 25-29.9.

8. The method of any of the above claims, wherein the patient is male human with a waist circumference exceeding 40 inches.

9. The method of any of the above claims, wherein the patient is a female human with a waist circumference exceeding 35 inches.

10. The method of any of the above claims, wherein the patient is at risk of further weight gain.

11. A method for inducing weight loss or preventing weight gain, comprising administering an effective amount of ITPP to a patient that:
has undertaken or will undertake a surgery of the digestive system;
is greater than about 80-100 pounds overweight;
has a BMI of greater than about 35; or
has a health problem related to obesity.

12. The method of claim 11, wherein the surgery of the digestive system is one or more of those classified under ICD-9-CM [42-54].

13. The method of claims 11 or 12, wherein the surgery of the digestive system is one or more of a restrictive surgery and a malabsorptive procedure.

14. The method of claim 13, wherein the restrictive surgery and/or malabsorptive procedure is one or more of vertical banded gastroplasty (VBG); gastric banding; sleeve gastrectomy; gastric bypass surgery, biliopancreatic diversion; and a cosmetic surgery.

15. The method of claim 14, wherein the gastric banding is LAP-BAND or REALIZE.

16. The method of claim 14, wherein the VBG is stomach stapling.

17. The method of claim 14, wherein the gastric bypass surgery is a Roux-en-Y gastric bypass.

18. The method of claim 14, wherein the cosmetic surgery is a liposuction.

19. The method of claim 18, wherein the liposuction is one or more of suction-assisted liposuction (SAL); ultrasound-assisted liposuction (UAL); power-assisted liposuction (PAL); twin-cannula (assisted) liposuction (TCAL or TCL); external ultrasound-assisted liposuction (XUAL or EUAL); water-assisted liposuction (WAL); laser assisted liposuction; tumescent liposuction; and cryolipolysis.

20. The method of claim 14, wherein the health problem related to obesity is selected from heart disease, sleep apnea, and high cholesterol.

21. The method of any of the above claims, wherein the patient is a man that is greater than 100 pounds overweight and/or has waist circumference exceeding 40 inches.

22. The method of any of the above claims, wherein the patient is a woman that is greater than 80 pounds overweight and/or waist circumference exceeding 35 inches.

23. A method for inducing weight loss or preventing weight gain, comprising administering an effective amount of ITPP to a patient in need thereof; wherein the patient does not substantially change caloric intake.

24. The method of claim 23, wherein the patient's caloric intake is not restricted by more than about 20%, or not by more than about 10%, or not by more than about 5% of the patient's caloric intake at the start of treatment.

25. The method of claims 23 or 24, wherein the patient's caloric intake is greater than about 2400 calories/day.

26. The method of any of the above claims, wherein the administration is any one of oral, intravenous, or parenteral.

27. The method of any of the above claims, wherein the effective amount is between about 0.2 and about 20 g/L when administered orally.

28. The method of any of the above claims, wherein the effective amount is about 4 g/mL, when administered intravenously.
